(19) United States
(12) Patent Application Publication
(10) Pub. No.: US 2015/0119853 A1
(43) Pub. Date: Apr. 30, 2015

(54) Convertible Shape Catheter And Method Of Use

(71) Applicant: MedWerks, LLC, St. Paul, MN (US)

(72) Inventors: John P. Gainor, Mendota Heights, MN (US); Robert F. Wilson, Roseville, MN (US); Uma S. Valeti, St. Paul, MN (US)

(21) Appl. No.: 14/527,734

(22) Filed: Oct. 29, 2014

Related U.S. Application Data
(60) Provisional application No. 61/897,145, filed on Oct. 29, 2013.

(51) Int. Cl. A61M 25/00; A61M 25/01

(52) U.S. Cl. CPC A61M 25/0041; A61M 25/0169
USPC 604/508

(57) ABSTRACT

A method of controlling and changing the shape of a catheter in situ that includes the use of two nested catheters. The catheters have two different shapes that may add, cancel, or override each other when one catheter is placed within the other. The shape may be changed by advancing or rotating one catheter relative to the other catheter.

### RELATED APPLICATIONS

This application claims priority to U.S. Provisional Application Ser. No. 61/897,145 filed Oct. 29, 2013 entitled Convertible Shape Catheter And Method Of Use, which is hereby incorporated herein by reference in its entirety.

### FIELD OF THE INVENTION

The present invention relates to the field of medical catheters. In particular, the present invention relates to catheters used to navigate vascular anatomy and cardiac structures.

### BACKGROUND OF THE INVENTION

There are a great number of medical conditions that necessitate physician intervention by catheter in order to provide diagnosis of or therapy for diseases of the vascular or cardiac systems. In many of the patients that require this type of treatment, the nature of their disease is such that their anatomy does not allow for easy or safe passage of catheters through the vasculature or to the target location. In these cases, a guidewire is often used to reach the target location followed by the catheter over that guidewire. However, in some cases the passage of the guidewire is also complicated by either the tortuosity of the vascular passage, the need to steer the guidewire or otherwise change the direction of advance in order to enter a selected bifurcation of the vessel or the desire to pass atraumatically through a cardiac structure such as a valve. In these cases, the guidewire is often advanced concurrently with a catheter of some type such as a coronary guide catheter or a pigtail catheter.

In the particular case of transcatheter aortic valve replacement, there is a need to place a relatively stiff guidewire across the native aortic valve in order to guide the placement of the valve delivery system. The native aortic valve is being treated because of a stenosis, or stiffening, of the native valve leaflets. This stenosis also makes it very difficult to advance a guidewire through the restricted valve. In order to pass the guidewire through the native valve, any number of catheters may be used in an attempt to orient the catheter tip in a way to allow the wire to pass through the restricted opening. In an ideal situation, a catheter called a pigtail catheter would be used to place the guidewire. The pigtail catheter is ideal because of its geometry—it is shaped in an approximately 270 degree curve such that the distal contact surface of the catheter is smooth and rounded, presenting an atraumatic surface of contact between the leading edge of the catheter and the cardiac structures that it contacts. It also provides an exit location for the guidewire that is not at the distal face of the catheter, minimizing the possibility that advancing the guidewire would perforate or otherwise damage structures upon its exit from the catheter. Unfortunately, the pigtail geometry that provides for safe advancement of the guidewire once it has passed through the aortic valve and into the left ventricle of the heart is often not the preferred geometry for orienting the tip of the guidewire for safe and effective aortic valve crossing. In many cases, a coronary guide catheter is needed for this purpose. One such curve, designated the AL1 coronary guide curve, is often used because it orients the tip of the catheter in a generally axial direction and the catheter allows for some steering of the guidewire tip to align the wire with the center of the valve. This greatly improves the likelihood of success in crossing the native valve, but following the transition across the native valve, the guidewire remains oriented in the axial direction and increases the risk of left ventricular damage or perforation. The physician has to make a tradeoff between functionality while attempting to cross the native valve and the safety of the patient while advancing the guidewire once the catheter is across the valve.

### SUMMARY OF THE INVENTION

According to one aspect of the invention, a catheter is disclosed that can be converted from one preferred shape into another preferred shape by means of changing the axial alignment or radial orientation between two slidably disposed component members. In the case of a catheter that is used to cross a stenotic native aortic valve, one catheter component may be shaped in the AL1 configuration or another preferred shape in order to effect crossing of the stenotic valve with a guidewire. Once the tip of the wire has crossed the valve, the second catheter component may be advanced to configure the catheter construct into a pigtail catheter shape in order to provide a safe geometry for further advancement of the guidewire into the left ventricle and appropriate placement of the wire along the ventricular wall.

Alternately, the device can be configured in such a way that when the two catheter components are aligned longitudinally, the combination of the catheter shapes result in an AL1 or first preferred configuration. The retraction of one of the components, either the inner or outer catheter, would result in the remaining distal catheter component recovering to a pigtail or second preferred configuration.

In yet another embodiment, the two catheter components can be configured such that when the catheters are aligned longitudinally and held in a first radial orientation, the shape and stiffness of the two components result in a first preferred configuration. Rotation of either the inner or outer component to a second radial orientation changes the interactions between the two catheter component shapes with a resultant second configuration.

There are numerous means by which the two catheter components may be configured to provide a first and second preferred shape. In one embodiment the second catheter component is substantially stiffer than the first catheter component when the first catheter component is shaped in the first configuration and the second catheter is shaped in the second configuration. When the second catheter component is advanced over the first catheter component, the shape of the first catheter component is overwhelmed by the second catheter component and the combined construct substantially takes the shape of the second catheter component.

In another embodiment, the first catheter component is substantially stiffer than the second catheter component when the second catheter component is shaped in the first configuration and the first catheter component is shaped in the second configuration. When the first catheter component is advanced within the second catheter component, the shape of the second catheter component is overwhelmed by the first catheter component and the combined construct substantially takes the shape of the first catheter component.

In yet another embodiment, the lengths of the catheter components are substantially different such that advancement of one catheter component over or within the other catheter component removes the other catheter component from the effective distal end of the catheter. This allows the distal shape of the most distal catheter component to take a predetermined shape independent of the configuration of the proximal catheter shape.

In another embodiment, the catheter components are constructed with regions of variable stiffness. Relative axial alignment of the regions of greater or lesser stiffness allow the combination catheter to take on different shapes as necessary. When one catheter component with a minimally stiff regions is aligned with the second catheter component with minimally stiff regions, a configuration that provides one extreme of shape can be achieved due to the relatively flexible regions of the combined catheter. Using the same two catheter components, but with the regions of maximal stiffness on one catheter component bridging the minimally stiff regions of the second catheter, a much stiffer combined construct can be achieved and a second extreme of shape can be achieved. By modulating the engagement of the relatively stiff regions of one catheter component with those of the second catheter component, a range of combined catheter shapes can be achieved through axial manipulation of the catheter components with respect to one another.

In another embodiment, the shape of the combined catheter can be modulated by radial manipulation of one catheter component relative to the other, with or without axial adjustment of catheter component position. Two catheter components each constructed with a distal shape can provide a range of combined catheter shapes through variable rotational alignment of the two catheter components. The range of combined device shapes can vary widely depending upon the geometry and relative stiffness of the two catheter components.

In any of these embodiments, the dual catheter design has uses beyond simply crossing the native valve. With the distal catheter component in the left ventricle, the second catheter may be placed with the distal tip just proximal to the native valve and a dual-pressure measurement may be taken from the proximal hubs of each of the catheters. In this manner, a direct pressure gradient may be measured across a stenotic valve or a prosthetic valve replacement.

While these device descriptions are focused on therapy related to the aortic valve, a combination catheter as described has applications for many other diagnostic and therapeutic catheterization purposes. A combination catheter of this type may be used to provide guide and support for access to the coronary arteries, the renal arteries, crossing lesions or restrictions in the cardiac, neurovascular or peripheral vasculature, or any other application in which a first preferred catheter configuration is required for a first purpose while a second preferred catheter configuration is required for a second purpose and the exchange of catheters causes procedural delay or exposes the patient to additional risk.

### BRIEF DESCRIPTION OF THE DRAWINGS

FIG. 1 depicts an embodiment of a catheter of the invention in the heart;

FIG. 2 depicts an embodiment of a catheter of the invention across the native aortic valve;

FIG. 3 depicts an embodiment of a pigtail catheter of the invention being passed through an embodiment of an AL1 catheter of the invention and over an embodiment of a guidewire of the invention to enter the left ventricle;

FIG. 4 depicts an embodiment of a pigtail catheter of the invention being advanced into the apex of the left ventricle;

FIG. 5 depicts an embodiment of a guidewire of the invention being laid into the left ventricle in a preferred configuration with a loop crossing the ventricular apex, and with an embodiment of a pigtail catheter of the invention backed up towards the native aortic valve;

FIGS. 6a and 6b are an image series of an embodiment of a combined catheter of the invention used in an embodiment of an AL1 configuration of the invention, with retraction of an outer component resulting in an embodiment of a pigtail configuration of the invention;

FIGS. 7a and 7b are an image series of an embodiment of a combined catheter of the invention used in an embodiment of an AL1 configuration of the invention, with retraction of an inner component resulting in an embodiment of a pigtail configuration of the invention; and,

FIGS. 8a and 8b are an image series of an embodiment of a combined catheter of the invention, with full retraction of one catheter component providing a first preferred shape while full retraction of a second catheter component provides a second preferred shape.

### DETAILED DESCRIPTION OF THE DRAWINGS

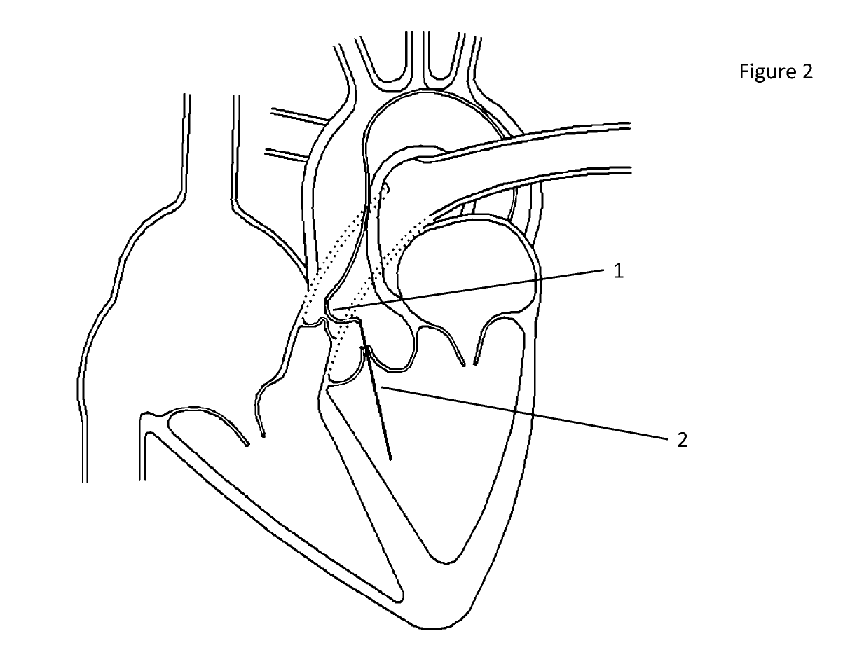

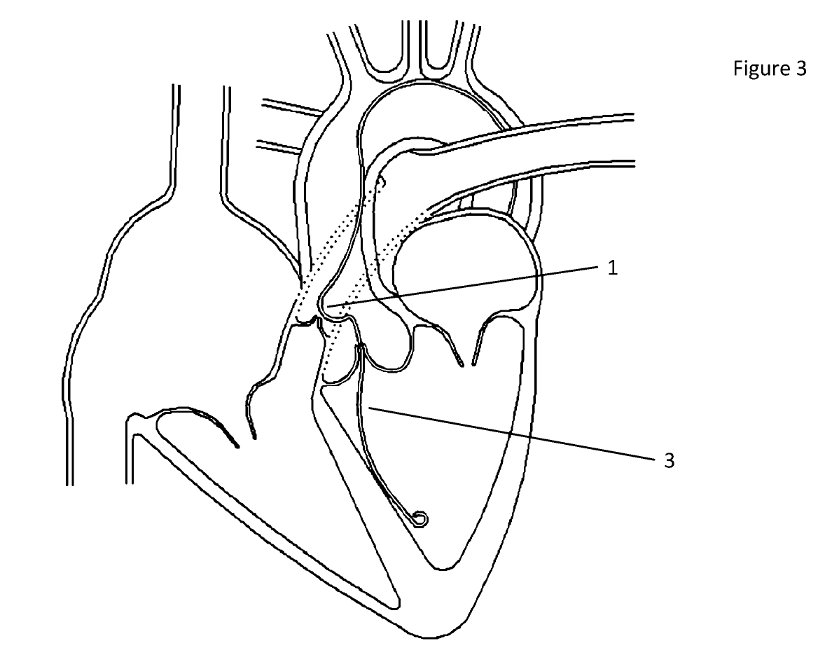

Referring now to the figures, the invention generally comprises a kit of components that may include a first catheter component 1, a guidewire 2 (see e.g. FIG. 2) and/or a second catheter component 3 (see e.g. FIG. 3). The first catheter 1 may be shaped to function as an AL 1 component. The guidewire 2 may be a variety of guidewire embodiments, including, but not limited to straight-tipped or non-straight-tipped, such as J-tipped and the like. The second catheter 3 may be shaped to function as a pigtail catheter.

The two catheters 1 and 3 are sized such that one catheter fits within the other. In some embodiments, catheter 1 fits within catheter 3 and in other embodiments, catheter 3 fits within catheter 1. The two catheters have different shapes. In one embodiment, catheter 1 is an AL1 shaped catheter and catheter 3 is a pig-tail shaped catheter. However, because one catheter fits within the other, the shapes may override each other or combine to form new shapes. The degree to which the shapes override each other, or combine to form new shapes, is dependent on the stiffness or softness of the catheters relative to each other. Moreover, it is envisioned that unique and desired shapes could be attained by providing catheters having variable stiffnesses. Additionally, the effect that the two catheters 1 and 3 have on each other may be varied not only depending on how far one catheter is inserted into the other, but also on the radial orientation of one catheter relative to each other.

As such, methods are described herein involving attaining desired shapes by selecting two catheters 1 and 3 having different shapes, and manipulating the catheters both axially and radially relative to each other at various stages in the advancement of one or both of the catheters into a desired target location, such as the heart. In at least one embodiment, a guidewire is also used to place the catheters in the target location.

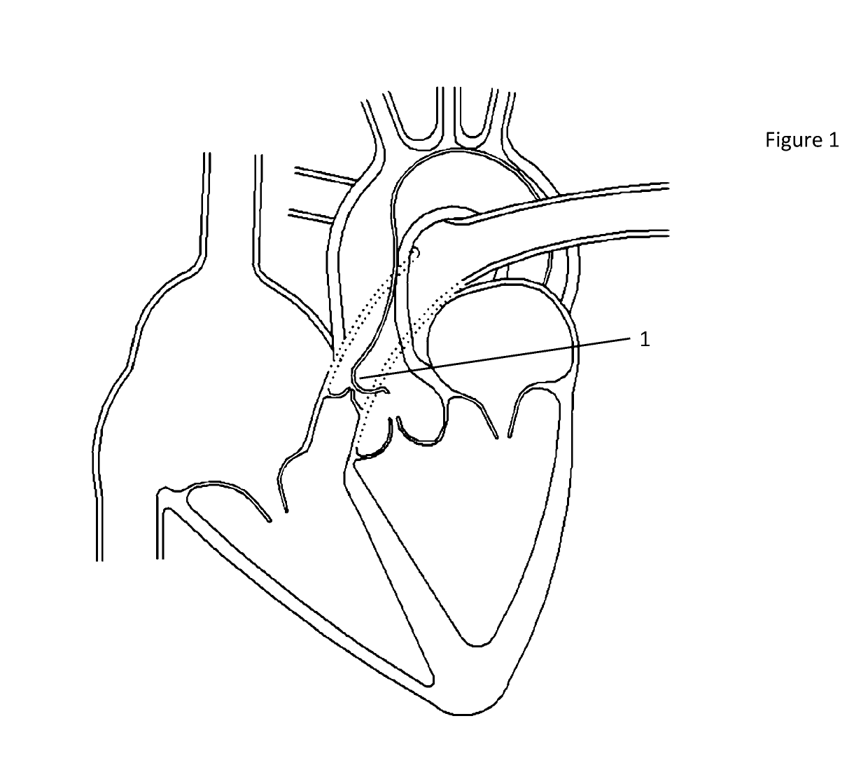

By way of non-limiting example, FIG. 1 depicts the placement of an AL1 shaped catheter component 1 near the aortic valve. The pulmonary artery has been hidden from view to show the placement of the AL1 catheter component 1 relative to the native valve. The placement of catheter 1 may have been achieved using known techniques.

Next, FIG. 2 depicts the passage of a straight-tipped guidewire 2 through the AL1 catheter component 1 and across the native aortic valve and into the left ventricle LV.

In one embodiment, the guidewire 2 is left in place and, as shown in FIG. 3, a catheter 3, in this case a pigtail catheter, has been advanced through catheter 1, but over the guidewire 2 (not seen in FIG. 30, to a preferred placement in the LV.

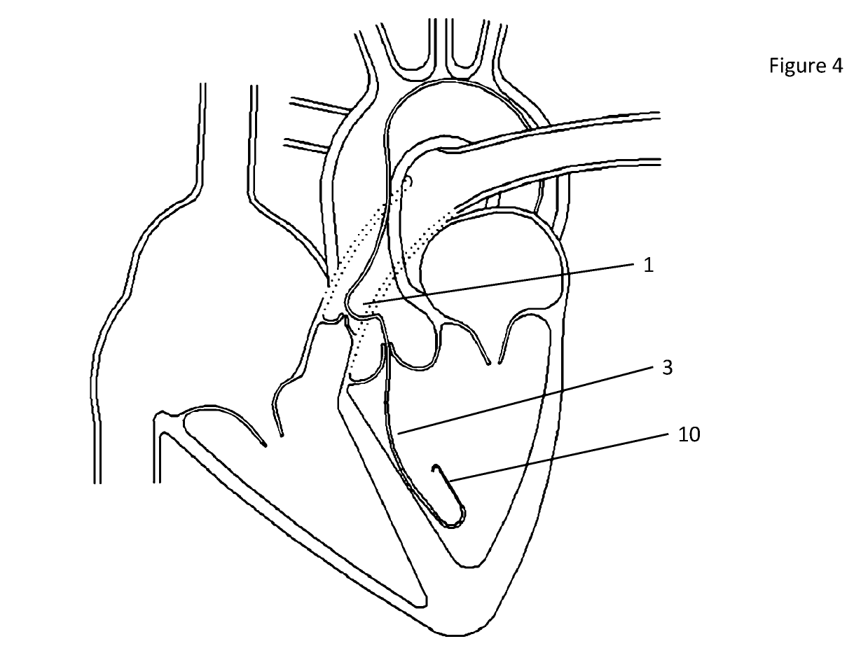

FIG. 4 depicts the advancement of a J-tipped guidewire 10 through the pigtail catheter component 3. As the guidewire 10 exits the pigtail catheter component 3, it moves in a direction towards the aortic valve and away from the apex of the left ventricle.

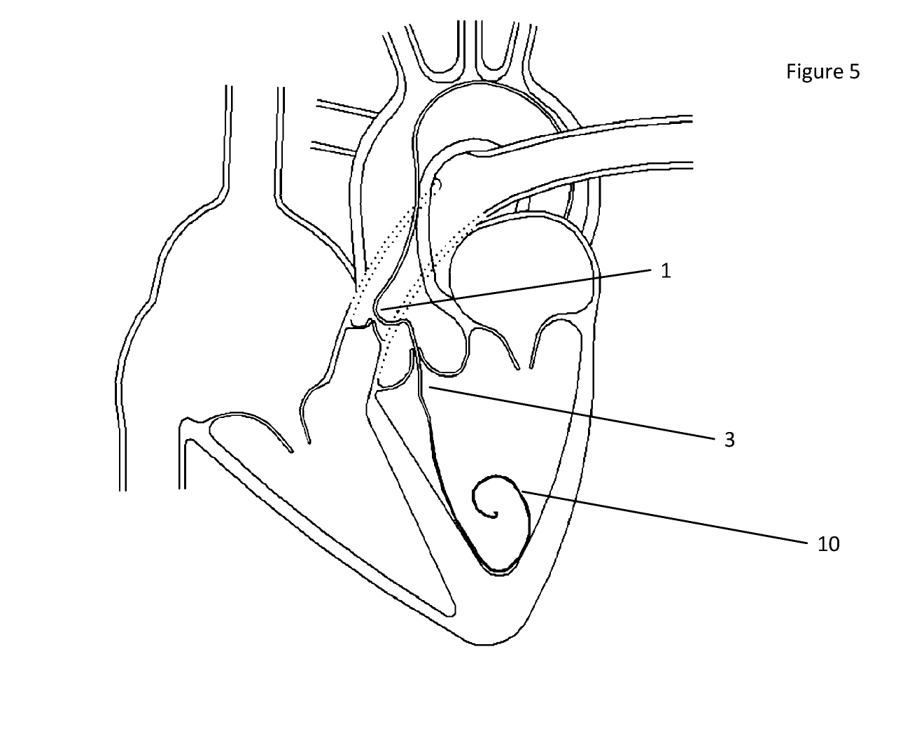

FIG. 5 depicts the placement of the guidewire 10 in preferred position along the wall of the left ventricle. This placement is guided by advancing the guidewire 10 while retracting the pigtail catheter component 3 to allow the guidewire loop to take shape.

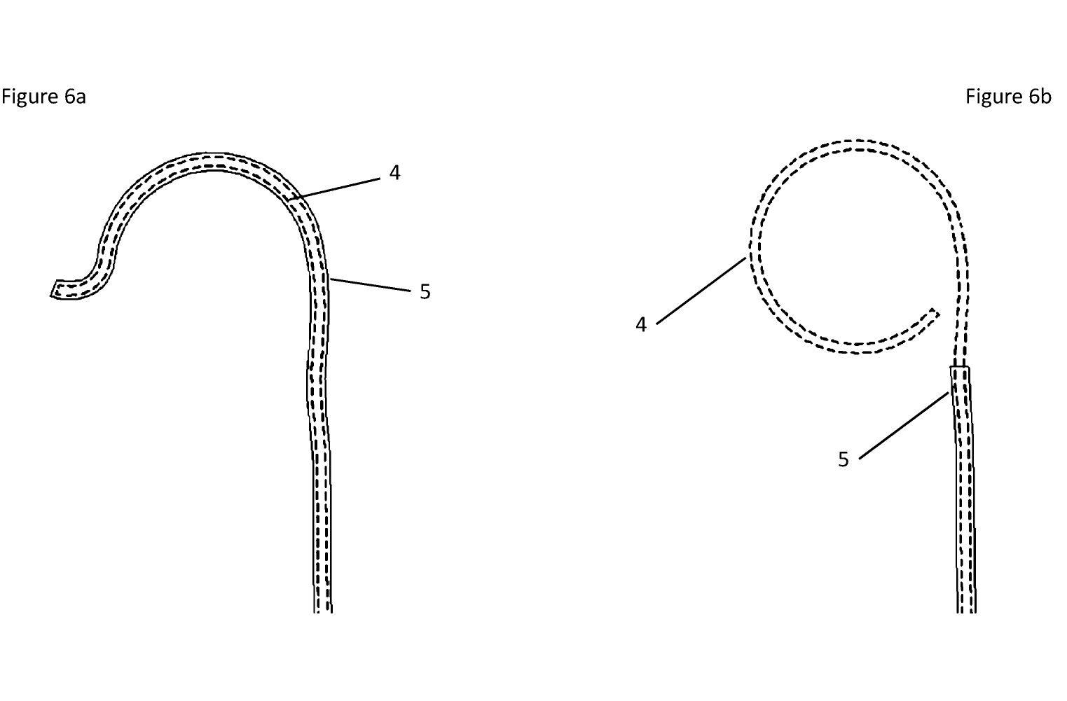

FIGS. 6a and 6b show the influence one catheter can have on the other. FIG. 6a depicts a catheter system design in which the combined shapes of the inner 4 and the outer 5 catheter components take on a first shape when the catheter components 4 and 5 are axially aligned. FIG. 6a shows the inner catheter 4 and the outer catheter 5 aligned axially, taking on an AL1 shape. FIG. 6b shows the outer catheter 5 retracted, allowing the inner catheter 4 to take on a pigtail catheter shape. Numbers 4 and 5 are used here instead of numbers 1 and 3 to indicate that this effect may be achieved by inserting catheter 1 into catheter 3 and vice versa. The resulting shape, as described above, depends on the relative catheter stiffnesses and radial and axial orientations.

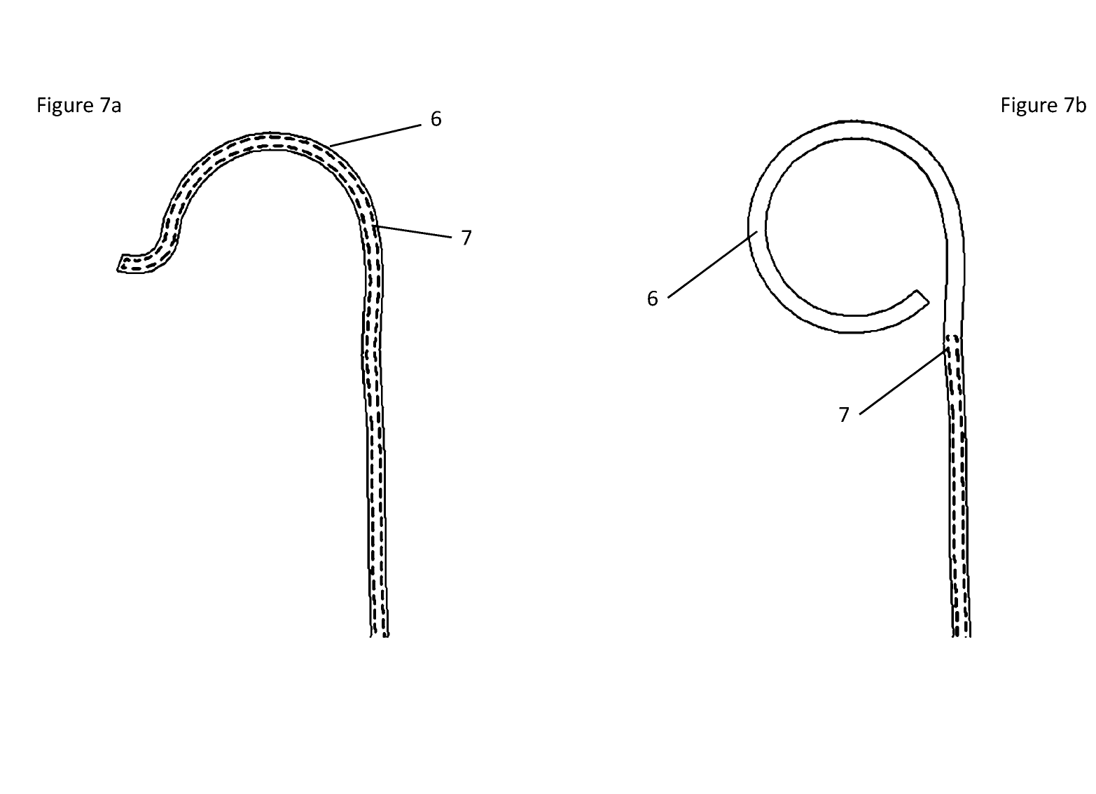

FIGS. 7a and 7b depict a catheter system design in which the combined shapes of the inner and the outer catheter components take on a first shape when the catheter components are axially aligned. FIG. 7a shows the inner catheter 7 and the outer catheter 6 aligned axially, taking on an AL1 shape. FIG. 7b shows the inner catheter 7 retracted, allowing the outer catheter 6 to take on a pigtail catheter shape.

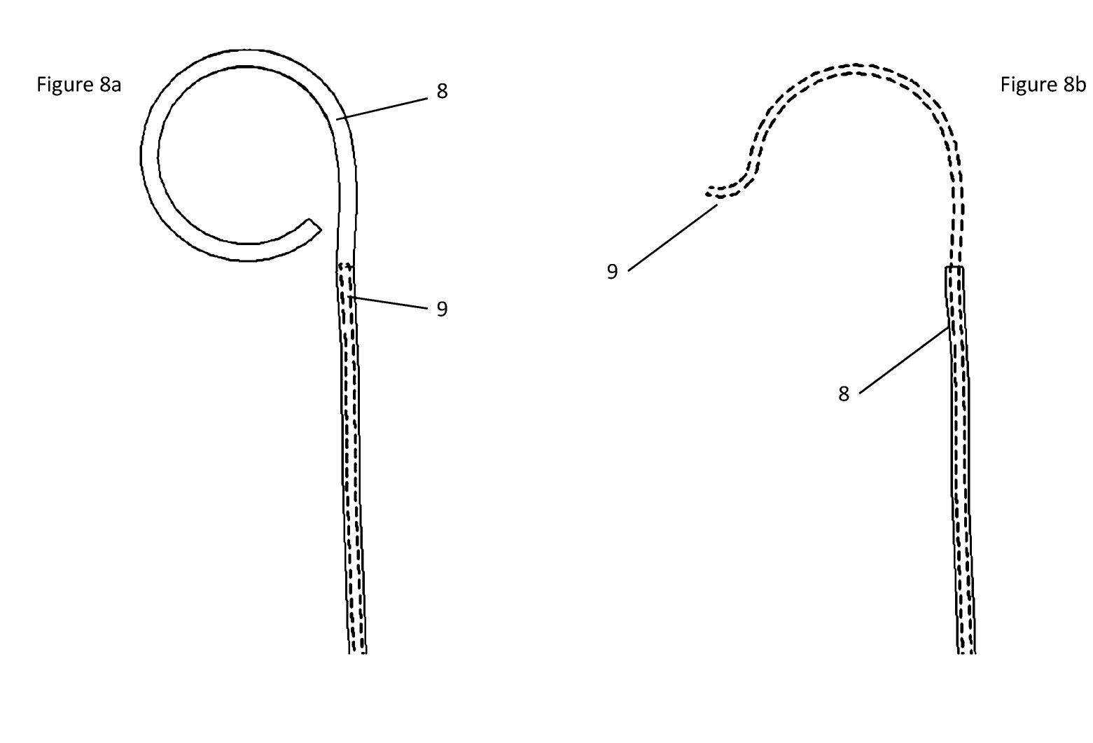

FIGS. 8a and 8b depict a catheter system design in which each catheter component takes on a preferred shape independent of the other catheter component. FIG. 8a shows an outer catheter 8 with a pigtail shape while the inner catheter 9 is retracted. FIG. 8b shows an inner catheter 9 with an AL1 shape when the outer catheter 8 is retracted.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

We claim:

1. A method of controlling a shape of a catheter comprising:
providing a first catheter having a first shape;
inserting a second catheter having a second shape into the first catheter, thereby changing the shape of at least one of the first catheter and the second catheter.

2. The method of claim 1 wherein providing a first catheter having a first shape comprises providing a first catheter having an AL1 shape.

3. The method of claim 1 wherein providing a first catheter having a first shape comprises providing a first catheter having a pigtail shape.

4. The method of claim 1 wherein providing a second catheter having a second shape comprises providing a second catheter having an AL1 shape.

5. The method of claim 1 wherein providing a second catheter having a second shape comprises providing a second catheter having a pigtail shape.

6. The method of claim 1 wherein providing a second catheter comprises providing a second catheter having a stiffness different than a stiffness of the first catheter.

7. The method of claim 1 further comprising rotating said second catheter relative to said first catheter to change a shape of at least one of the first catheter and the second catheter.

8. A method of placing a catheter into a desired location in a patient comprising:
navigating a first catheter to a location proximal of the desired location;
advancing a second catheter through a lumen of the first catheter until a distal end of the second catheter is in the desire location;
wherein the first catheter has a different shape than said second catheter.

9. The method of claim 8 wherein when said second catheter is inside said first catheter, said second catheter assumes the shape of said first catheter.

10. The method of claim 8 wherein said first catheter has an AL1 shape.

11. The method of claim 8 wherein said first catheter has a pigtail shape.

12. The method of claim 8 wherein the second catheter has an AL1 shape.

13. The method of claim 8 wherein the second catheter has a pigtail shape.

14. The method of claim 8 further comprising, advancing a guidewire through said first catheter before the step of advancing a second catheter through a lumen of the first catheter.

15. The method of claim 14 wherein the step of advancing a second catheter through a lumen of the first catheter comprises advancing the second catheter over the guidewire and through the first catheter.

16. The method of claim 8 wherein the step of advancing the second catheter through the first catheter causes the second catheter to assume the shape of the first catheter.

17. The method of claim 16 wherein the second catheter resumes an original shape after exiting a distal end of the first catheter.

18. The method of claim 8 wherein the first catheter has a stiffness that is different than a stiffness of the second catheter.

19. The method of claim 8 wherein at least one of the first and second catheter have axially varying stiffnesses.

20. The method of claim 8 further comprising advancing a guidewire through said second catheter after a distal end of the second catheter has reached a desired location.
